(19) United States
(12) Patent Application Publication
(10) Pub. No.: US 2018/0280628 A1
(43) Pub. Date: Oct. 4, 2018

(54) SYRINGE AND NEEDLE ASSEMBLY

(71) Applicants: LITE-ON ELECTRONICS (GUANGZHOU) LIMITED, GUANGZHOU (CN); Lite-On Technology Corporation, Taipei (TW)

(72) Inventors: Po-Yu Cheng, Taipei (TW); Yi-Feng Pu, Taipei (TW)

(73) Assignees: LITE-ON ELECTRONICS (GUANGZHOU) LIMITED, GUANGZHOU (CN); Lite-On Technology Corporation, Taipei (TW)

(21) Appl. No.: 15/939,266

(22) Filed: Mar. 28, 2018

(30) Foreign Application Priority Data
Mar. 29, 2017 (CN) 201710199002.1

(51) Int. Cl. A61M 5/32; A61M 5/315

(52) U.S. Cl. CPC A61M 5/3221; A61M 5/5066; A61M 2005/3223; A61M 5/31578

(57) ABSTRACT

A syringe and needle assembly includes an elongated hollow barrel, a needle unit, and a plunger assembly. The hollow barrel has a connecting portion. The needle unit is disposed at the connecting portion and has an engaging structure, and the engaging structure is located in the hollow barrel. A plunger rod of the plunger assembly is movably disposed in the hollow barrel and has a pushing portion and an operating portion formed in front and rear ends of the plunger rod respectively, and the pushing portion faces the connecting portion and has a plurality of elastic hooks. The plunger assembly is adapted to move towards the connecting portion to push a liquid in the hollow barrel to be injected through a needle of the needle unit, and the plunger assembly is adapted to keep moving towards the connecting portion, such that the elastic hooks are engaged with the engaging structure.

### CROSS REFERENCE TO RELATED APPLICATION

This application claims the priority benefit of China application serial no. 201710199002.1, filed on Mar. 29, 2017. The entirety of the above-mentioned patent application is hereby incorporated by reference herein and made a part of specification.

### BACKGROUND OF THE INVENTION

### 1. Field of the Invention

The invention relates to assemblies of syringes and needles. More particularly, the invention relates to a syringe and needle assembly that includes a retractable needle.

### 2. Description of Related Art

Hypodermic syringes are widely used in the medical arts for administering medicaments and for drawing body fluid samples. A hypodermic syringe is generally a one-time use item and is discarded after being used for hygienic considerations and for preventing disease transmission. Nevertheless, as the needle of the hypodermic syringe is exposed to the outside environment after being used, the needle is thus susceptible to be in contact with a physician who may thus be infected, or the hypodermic syringe may be repeatedly used owing to human errors, causing problems such as disease transmission. Accordingly, referring to FIG. 5, in some of the existing hypodermic syringes, an annular structure 310 is formed on a front end of a plunger and is engaged with an engaging structure 320 of a needle unit through an engaging hole 312 in the middle of the annular structure 310, such that the plunger can be pulled by the physician to retract the needle unit into the syringe body. Nevertheless, the annular structure 310 has a poor elastic deformation capability, and the engaging structure of the needle unit cannot be smoothly engaged into the engaging hole at the middle of the annular structure 310, as such, a user needs to apply a stronger external force needs to push the engaging structure 320 of the needle unit into the engaging hole 312 at the middle of the annular structure 310. Moreover, the annular structure 310 is not easy to be demolded during manufacturing, and a problem such as excessive manufacturing tolerance may be generated.

### SUMMARY OF THE INVENTION

The invention provides a syringe and needle assembly in which a retractable needle unit can be withdrawn into a syringe barrel after an injection, so as to prevent inadvertent contact with the contaminated needle during subsequent handling of the used syringe.

The invention provides a syringe and needle assembly having a simple and secure structure and is easy to be manufactured.

In an embodiment of the invention, the syringe and needle assembly includes an elongated hollow barrel, a needle unit, and a plunger assembly. The elongated hollow barrel includes proximal and distal ends, an interior wall, and openings at the proximal and distal ends. The distal end of the elongated hollow barrel defines a connecting portion at the opening of the distal end. The needle unit includes a hollow needle, a hub, and an engaging structure. The hub having an interior passageway is removably held in the opening of the distal end of the elongated hollow barrel through the connecting portion, the engaging structure is located in the elongated hollow barrel, and the hollow needle is mounted in the hub and the engaging structure. The plunger assembly is axially displaceable within the elongated hollow barrel, and includes an elongated plunger rod, a pushing portion formed in the front end of the plunger rod and an operating portion formed in the rear end of the plunger rod. The pushing portion facing the connecting portion has a plurality of elastic hooks, and the elastic hooks are separated from each other and are disposed correspondingly to the engaging structure.

The plunger assembly is adapted to move from a separating state towards the connecting portion to a first position for pushing a liquid contained in the elongated hollow barrel to pass through the hollow needle, and the plunger assembly is adapted to move towards the connecting portion to a second position such that the elastic hooks are fully engaged with the engaging structure. The plunger assembly is also adapted to move in a direction away from the connecting portion so that an associated force can be applied to the needle unit after the elastic hooks are engaged with the engaging structure, as the needle unit being draw back into the elongated hollow barrel.

In the syringe and needle assembly according to an embodiment of the invention, the engaging structure includes a first guiding inclined surface that extends downwards along an edge of a first engaging surface of the engaging structure that faces the connecting portion, and a radial width of the engaging structure gradually reduces in a direction away from the connecting portion. Moreover, each of the elastic hooks includes an elastic arm portion and an engaging portion, a spacing distance between a pair of the opposed engaging portions is less than a radical width of the engaging structure. The engaging portion further include a second guiding inclined surface and a second engaging surface. Size of the second engaging surface of the elastic hook may be greater than or equal to size of the first engaging surface of the engaging structure. When each of the engaging portions is in contact with the engaging structure and the plunger assembly keeps moving towards the connecting portion, each of the elastic arm portions is elastically deformed outwardly until each of the engaging portions crosses over the engaging structure and fully engages with the first engaging surface of the engaging structure. When each of the second guiding inclined surfaces is in contact with the engaging structure and the plunger assembly keeps moving towards the connecting portion, each of the elastic hooks is deformed smoothly until cross over and to engage with the engaging structure as guided by the second guiding inclined surface together with the first guiding inclined surface of the engaging structure.

In the syringe and needle assembly according to an embodiment of the invention, the number of the elastic hooks is two, and the two elastic hooks are respectively located at two opposite sides of the engaging structure when the two elastic hooks are engaged with the engaging structure. When each of the engaging portions is in contact with the engaging structure and the plunger assembly keeps moving towards the connecting portion, each of the engaging portions crosses over the engaging structure and fully engages with the first engaging surface of the engaging structure.

In the syringe and needle assembly according to an embodiment of the invention, the syringe and needle assembly may further include an elastic sealing ring which is sleeved on the pushing portion of the plunger assembly and fits snugly against an interior of the elongated hollow barrel and serves to force a liquid contained in the interior of the elongated hollow barrel out through the hollow needle. At least a portion of each of the elastic hooks is located outside the elastic sealing ring. Alternatively, outer surfaces of the elastic hooks are entirely surrounded by the elastic sealing ring, and the elastic sealing ring has an extending portion located between the elastic hooks, and the elastic hooks pass through the extending portion to at least partially locate outside the extending portion; thus, when the plunger assembly moves towards the connecting portion to push the liquid contained in the elongated hollow barrel to be injected through the hollow needle, the engaging structure can be stopped at the extending portion, and the plunger assembly is adapted to keep moving towards the connecting portion such that the engaging structure compresses the extending portion and the elastic hooks are engaged with the engaging structure.

In an embodiment of the invention, the syringe and needle assembly may further include an elastic filling ring sleeved on the protruding portion of the hub and fitting snugly against an interior of the elongated hollow barrel. When the plunger assembly is moved towards the connecting portion to push the liquid contained in the elongated hollow barrel to be injected through the hollow needle, the elastic filling ring is stopped by the plunger assembly, and the plunger assembly is adapted to keep moving towards the connecting portion against an elastic restoring force of the elastic filling ring and compress the elastic filling ring, such that the elastic hooks are tightly engaged with the engaging structure. In addition, the plunger assembly may include a stopping portion connected to the plunger rod, and when the plunger assembly moves in the direction away from the connecting portion to draw the needle unit to retract into the elongated hollow barrel, the stopping portion is abutted against an outer edge of the elongated hollow barrel to prevent the plunger assembly from moving towards the connecting portion.

To sum up, in the syringe and needle assembly provided by the embodiments of the invention, the plunger assembly can be moved towards the connecting portion of the elongated hollow barrel after the injection, so that the elastic hooks of the plunger assembly are engaged with the engaging structure of the needle unit, and then the plunger assembly can be moved in the direction away from the connecting portion to retract the hollow needle unit into the elongated hollow barrel. Therefore, the hollow needle of the syringe and needle assembly, after being used, is not exposed outside the elongated hollow barrel, so that the hollow needle can be prevented from infecting the physicians due to inadvertent contacts, or the syringe and needle assembly can be prevented from being reused owing to human errors which may result in problems such as disease transmission. In addition, the syringe and needle assembly provided by the embodiments of the invention is engaged with the engaging structure of the needle unit through the plurality of separate elastic hooks, rather than being engaged with the engaging structure of the needle unit through the engaging hole at the middle of the annular structure as did in conventional syringe and needle assembly. Therefore, each of the elastic hooks, with its favorable elastic deformation capability, can be engaged smoothly with the engaging structure. Moreover, the elastic hooks, as being separated from each other, are easy to be demolded during manufacturing, and thus problems such as excessive manufacturing tolerance can be prevented.

To make the aforementioned and other features and advantages of the invention more comprehensible, several embodiments accompanied with drawings are described in detail as follows.

### BRIEF DESCRIPTION OF THE DRAWINGS

The accompanying drawings are included to provide a further understanding of the invention, and are incorporated in and constitute a part of this specification. The drawings illustrate embodiments of the invention and, together with the description, serve to explain the principles of the invention.

FIG. 1A is a three-dimensional diagram illustrating a plunger assembly and a needle unit of a syringe and needle assembly, in a state prior to use, according to an embodiment of the invention.

FIG. 1B is a three-dimensional diagram illustrating the syringe and needle assembly of FIG. 1A after the completion of an injection, in which the plunger assembly is engaged with the needle unit.

FIG. 2A to FIG. 2E are enlarged cross-sectional views illustrating a process of operation for the syringe and needle assembly of FIG. 1A.

FIG. 3 is a three-dimensional diagram of an engaging structure of the syringe and needle assembly of FIG. 1A.

FIG. 4A to FIG. 4D are enlarged cross-sectional views illustrating a process of operation for a syringe and needle assembly according to another embodiment of the invention.

FIG. 5 is a schematic three-dimensional diagram of a conventional engaging structure of a needle unit and a conventional annular structure of a plunger.

### DESCRIPTION OF THE EMBODIMENTS

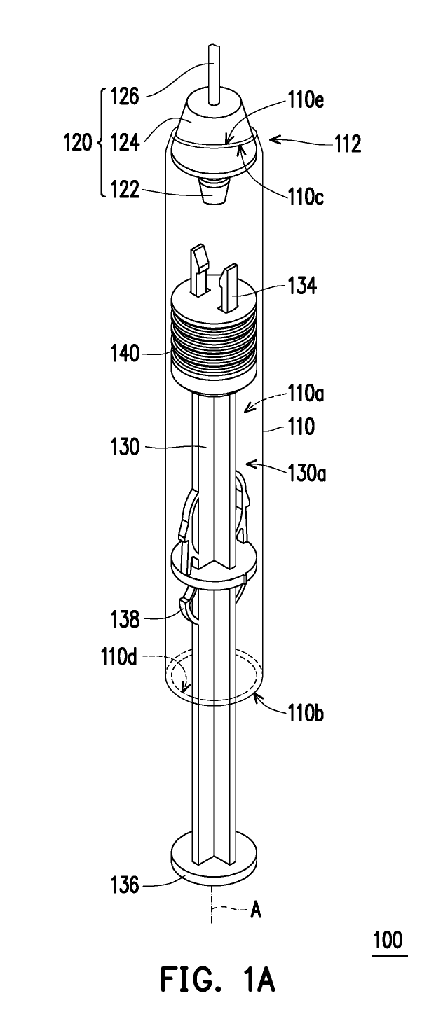

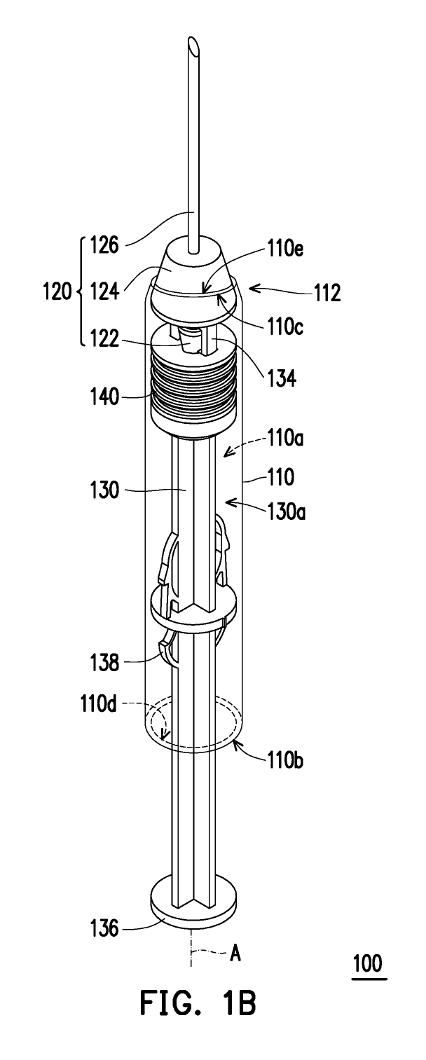

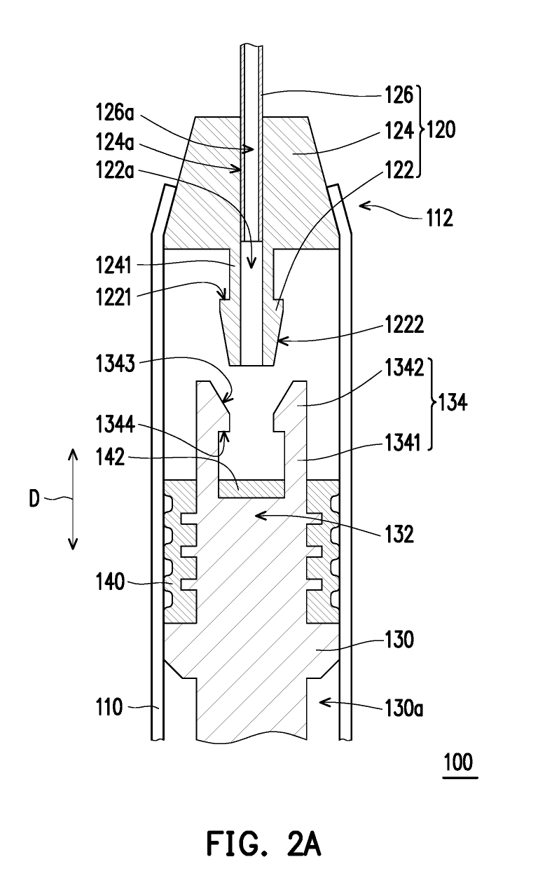

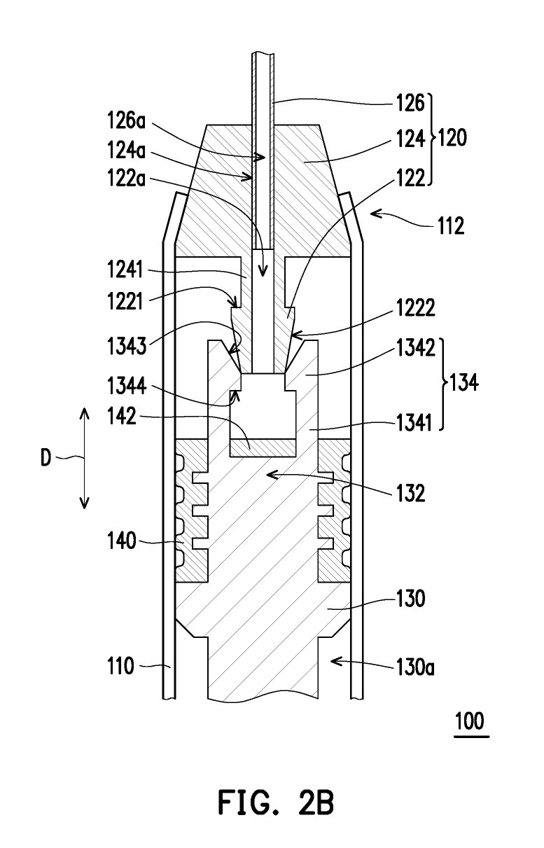

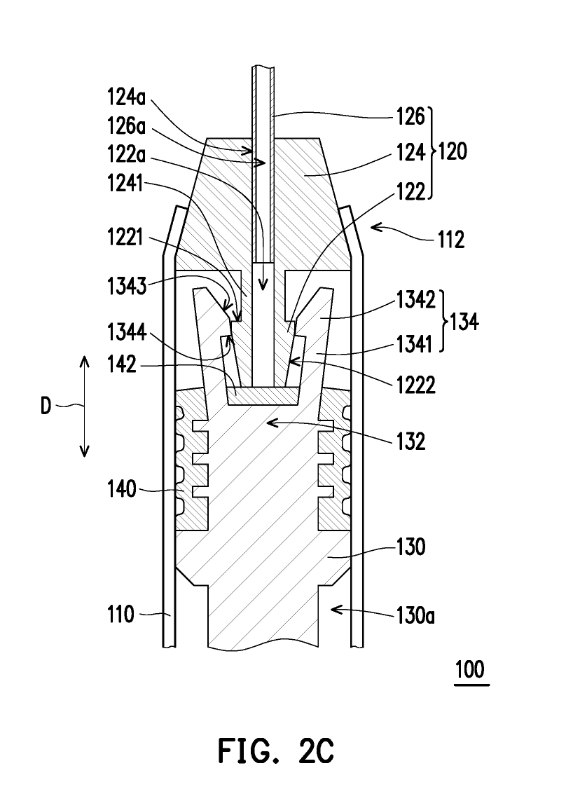

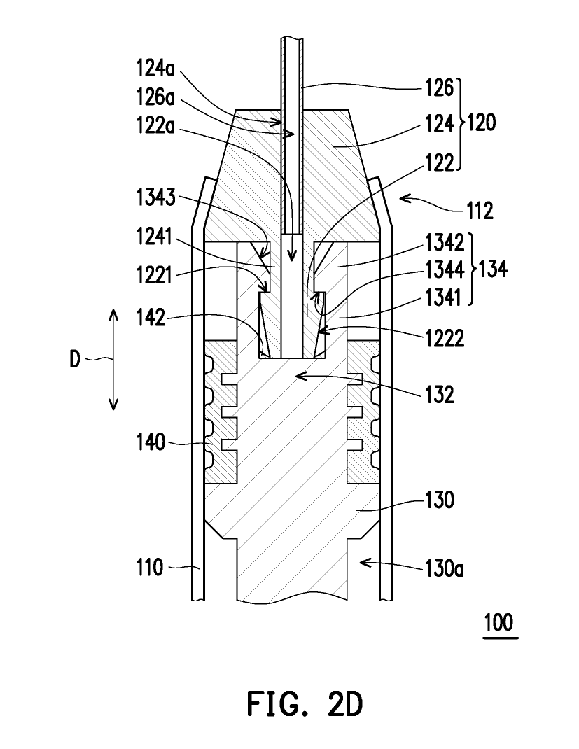

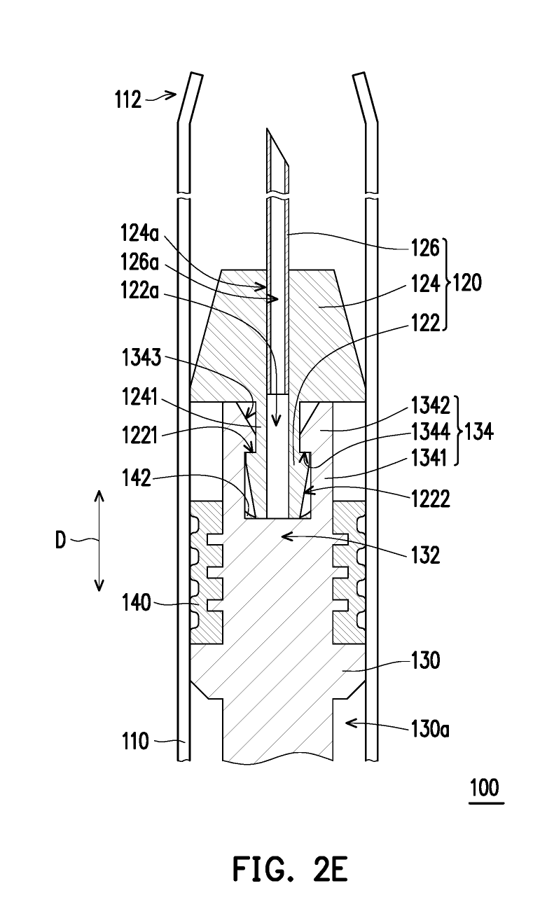

FIG. 1A is a three-dimensional diagram illustrating a plunger assembly and a needle unit of a syringe and needle assembly, in a state prior to use, according to an embodiment of the invention. FIG. 1B is a three-dimensional diagram illustrating the syringe and needle assembly of FIG. 1A after the completion of an injection, in which the plunger is engaged with the needle unit. FIG. 2A to FIG. 2E are enlarged cross-sectional views illustrating a process of operation for the syringe and needle assembly of FIG. 1A. FIG. 1B illustrates the plunger being engaged with the need unit in correspondence to the figure shown in FIG. 2D. Referring to FIG. 1A, FIG. 1B, and FIG. 2A, in the present embodiment, a syringe and needle assembly 100 is, for example, an injection syringe with a retractable needle, including an elongated hollow barrel 110, a needle unit 120, and an elongated plunger assembly 130. The hollow barrel 110 has an inside surface defining a chamber 110a, a proximal end 110b and a distal end 110c, and openings 110d, 110e at the proximal and distal ends 110b, 110c. In addition, the distal end 110c with the opening 110e at the distal end 110c defines a connecting portion 112 for connecting to the needle unit 120. The plunger assembly 130 can be inserted within the proximal end of the hollow barrel 110 and can be pushed into the interior of the hollow barrel 110 in order to pump fluid contained in the hollow barrel 110 out.

Specifically, in the present embodiment, the chamber is formed in the hollow barrel 110 for containing a liquid (e.g., an injection liquid), and the chamber includes a front chamber and a rear chamber. The needle unit 120 includes a hub 124 and a hollow needle 126 attached to the hub 124. The connecting portion 112 and the hub 124 together define an end of the front chamber of the hollow barrel 110. The hollow needle 126 is adapted for piercing into a body portion of a patient, and has a proximal end, a distal end and a lumen 126a therethrough. The hub 124 having an interior passageway 124a is removably held in the opening at the distal end of the hollow barrel 110 through the connecting portion 112. In this embodiment, an engaging structure 122 is connected to the hub 124 and is located in the front chamber of the hollow barrel 110. Preferably, the hub 124 includes a protruding portion 1241 (shown in FIG. 2A) extending radially outwardly from a bottom surface of the hub 124 to connect to the engaging structure 122. Preferably, the hub 124, the protruding portion 1241 and the engaging structure 122 are integrally formed. Alternatively, the hub 124, the protruding portion 1241 and the engaging structure 122 may be assembled together to form the needle unit 120. In addition, the engaging structure 122 includes an interior passageway 122a corresponding to the interior passageway 124a of the hub 124, and a first engaging surface 1221 which faces the connecting portion 112 of the hollow barrel 110. The proximal end of the hollow needle 126 is inserted into the interior passageway 124a of the hub 124 and passes completely through the hub 124 (and the engaging structure 122) so that the distal end of the hollow needle 126 is exposed and the lumen 126a of the hollow needle 126 is in fluid communication with the passageway 124a of the hub 124.

The plunger assembly 130 includes an elongated plunger rod 130a which is axially displaceable within the inner chamber 110a of the hollow barrel 110 and able to be moved back and forth. In this embodiment, a pushing portion 132 (shown in FIG. 2A) is formed in the front end of the plunger rod 130a and located in the chamber 110a of the hollow barrel 110. An operating portion 136 is formed in the rear end of the plunger rod 130a and located outside the hollow barrel 110 for a user to push or pull. In addition, the pushing portion 132 facing the connecting portion 112 has a plurality of elastic hooks 134. The elastic hooks 134 have opposed hook-shaped ends which are spaced from each other by a distance less than a diameter of the first engaging surface 1221. That is, a front end segment of the plunger assembly 130 includes the pushing portion 132, and a rear end segment of the plunger assembly 130 includes the operating portion 136.

The plunger assembly 130 of the present embodiment further includes a plunger seal 140, for example, an elastic sealing ring. The elastic sealing ring 140 can be sleeved on the pushing portion 132 of the plunger rod 130a through adhesion, structural engagement, or other suitable manners. The elastic sealing ring 140 fits snugly against the interior of the hollow barrel 110 and serves to force the liquid contents of the interior of the hollow barrel 110 (usually medication) out through the hollow needle 126. Since the elastic sealing ring 140 forms a fluid-tight seal with the interior of the hollow barrel 110, the elastic sealing ring 140 maintains airtightness between the pushing portion 132 of the plunger rod 130a and the needle unit 120 for containing the fluid medicine and ensuring that the fluid medicine in the hollow barrel 110 is injected only through the hollow needle 126 without leaking out unexpectedly from a rear chamber side of the hollow barrel 110. In the present embodiment, each of the elastic hooks 134 is at least partially located outside the elastic sealing ring 140, i.e., each of the elastic hooks 134 is not entirely encapsulated by the elastic sealing ring 140, and that each of the elastic hooks 134 can be elastically deformed smoothly.

In the operation of the syringe and needle assembly 100, by pulling the plunger assembly 130 in a D direction by the user, the pushing portion 132 of the plunger rod 130a moves away from the connecting portion 112, such that an external liquid (e.g., an injection liquid) is extracted and sucked through the hollow needle 126 into the inner chamber 110a of the hollow barrel 110. When the injection liquid is extracted and sucked to a predetermined dose, an injection operation can be executed. By pushing the plunger assembly 130 in a reverse direction of the D direction by the user, the pushing portion 132 of the plunger rod 130a is moved from a separating state shown in FIG. 2A towards the connecting portion 112 to a first position shown in FIG. 2C. The plunger assembly 130 pushes the liquid contained in the hollow barrel 110 to pass through the hollow needle 126 and to be injected. The first position, as shown in FIG. 2C, refers to that the elastic hooks 134 of the plunger rod 130a are pushed and deformed and thus are clamped outside the engaging structure 122 of the needle unit 120, and the elastic hooks 134 are not yet fully engaged with the engaging structure 122 at this time. Next, by continuously applying a force to the plunger assembly 130 by the user, the plunger assembly 130 keeps moving towards the connecting portion 112 to a second position shown in FIG. 2D. The second position refers to that the elastic hooks 134 of the plunger rod 130a are fully engaged with the engaging structure 122 of the needle unit 120. Then, by pulling the plunger assembly 130 by the user, the plunger assembly 130 moves in a direction away from the connecting portion 112. As the elastic hooks 134 and the engaging structure 122 are tightly engaged with each other, by pulling the plunger assembly 130, a force is thus applied together to the needle unit 120 at the same time, such that the needle unit 120 is drawn back into the chamber 110a of the hollow barrel 110 as shown in FIG. 2E.

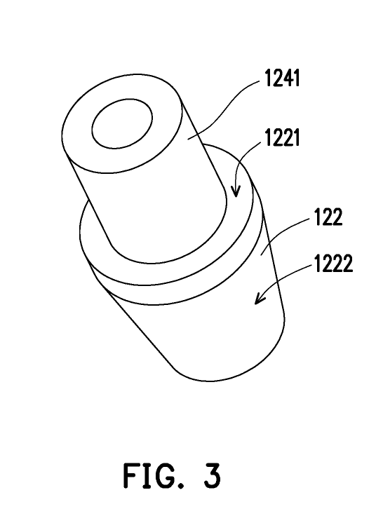

Specifically, the engaging structure 122 further includes a first guiding inclined surface 1222. The first guiding inclined surface 1222 extends downwards along an edge of the first engaging surface 1221, and a radial width of the engaging structure 122 gradually reduces in the direction away from the connecting portion 112. In the present embodiment, the engaging structure 122 can be a truncated cone structure. The first engaging surface 1221 of the engaging structure 122 is perpendicular to the moving direction D of the plunger assembly 130, such that the elastic hooks 134 of the plunger rod 130a can be securely engaged with the first engaging surface 1221. FIG. 3 is a three-dimensional diagram of the engaging structure shown in FIG. 1B. As shown in FIG. 3, the first engaging surface 1221 provided by the present embodiment is a continuous and annular flat surface. As such, regardless of how the plunger assembly 130 rotates along an axis A, the elastic hooks 134 can be in smooth contact with the first engaging surface 1221 in the second position (shown in FIG. 1B). In the present embodiment, a number of the elastic hooks is, for example, two, and the elastic hooks 134 are symmetrically disposed relative to each other. In the rest of the embodiments, the number of the elastic hooks 134 may be other suitable numbers, which should not be construed as a limitation to the invention.

As shown in FIG. 2A, each of the elastic hooks 134 provided by the present embodiment includes an elastic arm portion 1341 and an engaging portion 1342. The elastic arm portion 1341 is formed on a top surface of the pushing portion 132 and extends radially towards the connecting portion 112. The engaging portion 1342 is formed at an end (i.e., a free end) of the elastic arm portion 1341 and is shaped as a hook structure. When the plunger assembly 130 is moved to a state shown in FIG. 2B from an initial state shown in FIG. 2A and that each of the engaging portions 1342 are in contact with the engaging structure 122 and the plunger assembly 130 keeps moving towards the connecting portion 112, as a spacing distance between the two engaging portions 1342 is approximately less than a radial width of the engaging structure 122, such that each of the engaging portions 1342 are pushed by the engaging structure 122, and that the elastic arm portions 1341 are elastically deformed outwardly as shown in FIG. 2C until each of the engaging portions 1342 crosses over the engaging structure 122 and fully engages with the first engaging surface 1221 of the engaging structure 122 as shown in FIG. 2D. Moreover, each of the elastic hooks 134 also has a second engaging surface 1344. As shown in FIG. 2D, when the two elastic hooks 134 are engaged with the engaging structure 122, the elastic hooks 134 are respectively located at two opposite sides of the engaging structure 122, and the second engaging surfaces 1344 of the elastic hooks 134 are in contact with the first engaging surfaces 1221 of the engaging structure 122. In addition, in the present embodiment, sizes of the second engaging surfaces 1344 of the elastic hooks 134 may preferably be greater than or equal to sizes of the first engaging surfaces 1221 of the engaging structure 122. As such, the first engaging surfaces 1221 of the engaging structure 122 can completely lean against the second engaging surfaces 1344 of the elastic hooks 134, so as to achieve more secure engaging effect.

Specifically, the engaging portion 1342 of each of the elastic hooks 134 extends inwards towards the axis A to form a second guiding inclined surface 1343. When the second guiding inclined surface 1343 of each of the elastic hooks 134 is in contact with the first guiding inclined surface 1222 of the engaging structure 122 as shown in FIG. 2B, and the plunger assembly 130 keeps moving towards the connecting portion 112, each of the elastic hooks 134 is deformed smoothly until cross over and to engage with the engaging structure 122 as guided by the second guiding inclined surface 1343 together with the first guiding inclined surface 1222 of the engaging structure 122, as shown in FIG. 2C to FIG. 2D.

Furthermore, referring to FIG. 1A, FIG. 1B, and FIG. 2A, the elastic sealing ring 140 of the present embodiment includes an extending portion 142 that extends to between the elastic hooks 134 and surrounds the outer peripheral surface thereof. In other words, the elastic hooks 134 pass through, for example, corresponding holes on the extending portion 142 to at least partially locate outside the extending portion 142. When the plunger assembly 130 moves towards the connecting portion 112 to push the liquid in the hollow barrel 110 to be injected through the hollow needle 126, an end of the engaging structure 122 facing the plunger assembly 130 is stopped by the extending portion 142 of the elastic sealing ring 140, such that the plunger assembly 130 is prevented from being unexpectedly pushed by the user to the engaging state shown in FIG. 2D, i.e., the second position. When the user intends to have the elastic hooks 134 engaged with the engaging structure 122, a greater force needs to be applied to push the plunger assembly 130, such that the plunger assembly 130 keeps moving towards the connection portion 112 against an elastic restoring force of the extending portion 142. At the time, the engaging structure 122 can compress the extending portion 142, as shown in FIG. 2D, and each of the elastic hooks 134 is securely engaged with the engaging structure 122. Because of an elastic restoring force of the extending portion 142, the first engaging surface 1221 of the engaging structure 122 can be pushed for tightly engaging with the second engaging surface 1344 of the elastic hook 134 without loosening off easily.

In the present embodiment, referring to FIG. 1A and FIG. 1B, the plunger assembly 130 further includes a stopping portion 138 connected to the plunger rod 130a. In the operation of the syringe and needle assembly 100, when the plunger assembly 130 is pulled in the D direction by the user and the pushing portion 132 of the plunger rod 130a moves away from the connecting portion 112, such that the external liquid (e.g. fluid medicine) is extracted and sucked through the hollow needle 126 into the chamber 110a of the hollow barrel 110, the plunger rod 130a is prevented from being overly pulled out from the hollow barrel 110 by the user unexpectedly by the stopping portion 138 stopping at the open proximal end 110b of the hollow barrel 110. When the pushing portion 132 is engaged with the engaging structure 122 and the plunger assembly 130 moves in the direction away from the connecting portion 112 to draw the needle unit 120 to retract into the hollow barrel 110 after injection, the stopping portion 138 is pulled out from the hollow barrel 110, and the used need unit 120 is prevented from retracting to the connecting portion 112 unexpectedly by the user since the stopping portion 138 is abutted against an outer edge of the hollow barrel 110.

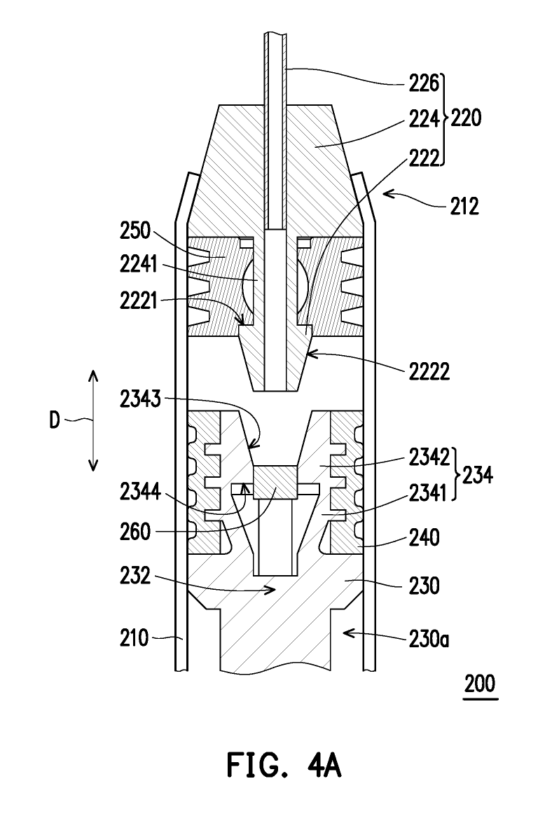

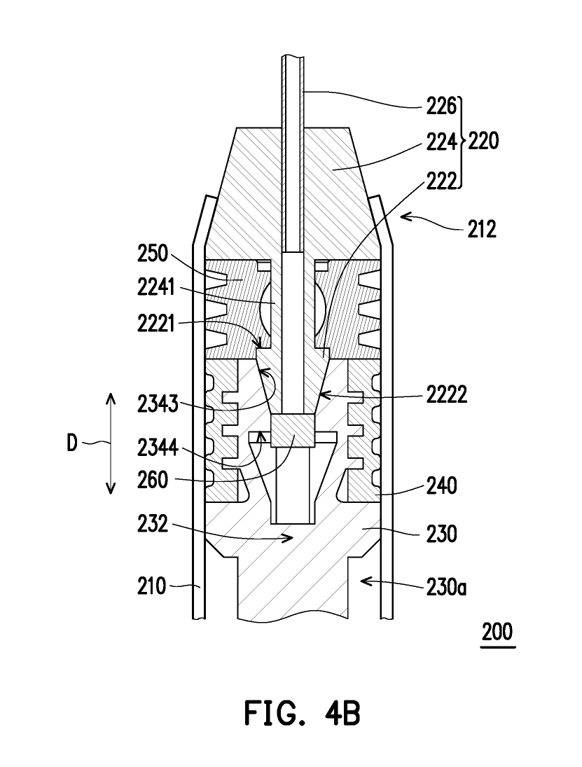

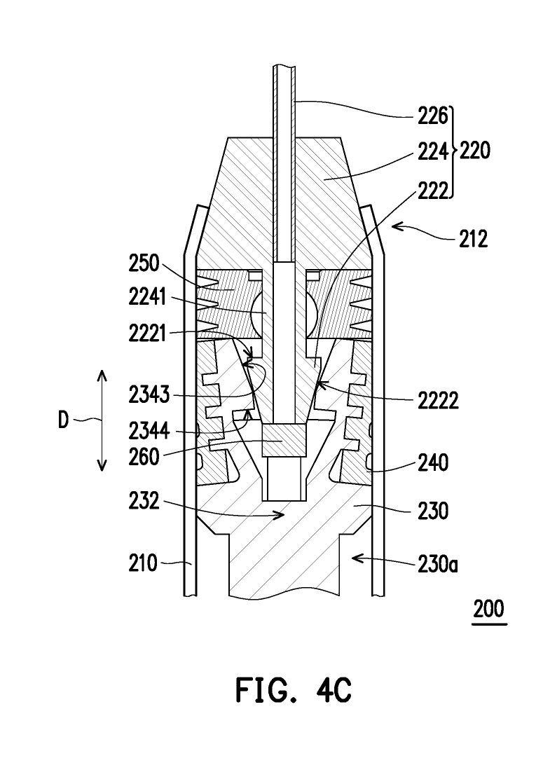

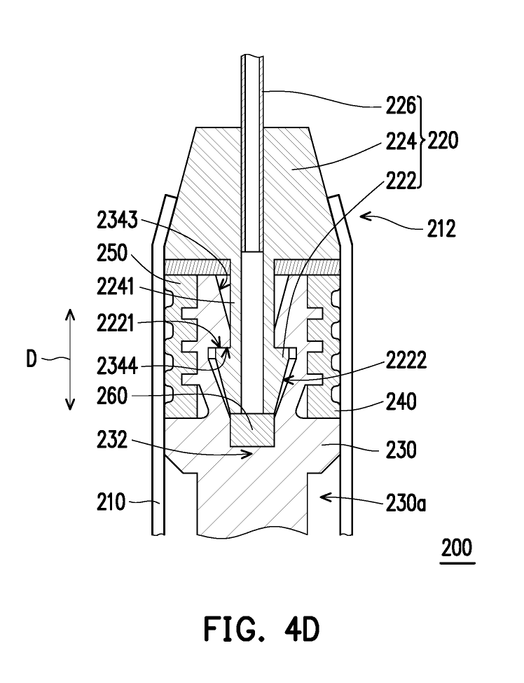

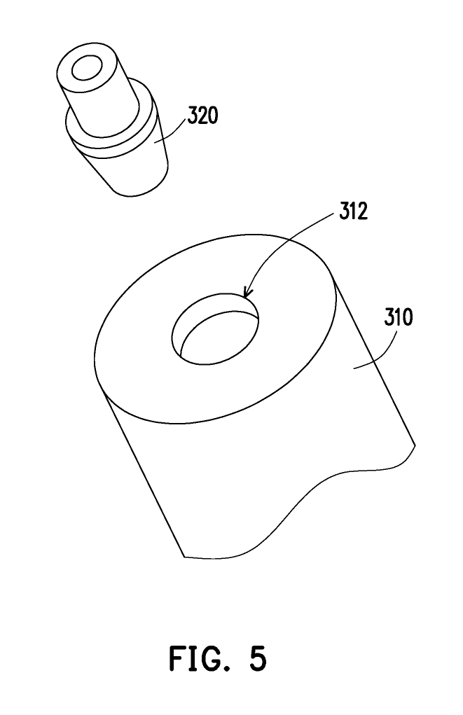

FIG. 4A to FIG. 4D are enlarged cross-sectional views illustrating a process of operation for a syringe and needle assembly according to another embodiment of the invention. In a syringe and needle assembly 200 of the present embodiment, a hollow barrel 210, a connecting portion 212, a needle unit 220, an engaging structure 222, a first engaging surface 2221, a first guiding inclined surface 2222, a hub 224, a protruding portion 2241, a needle 226, a plunger assembly 230, a pushing portion 232, elastic hooks 234, an elastic arm portion 2341, and engaging portion 2342, a second guiding inclined surface 2343, and a second engaging surface 2344 are disposed and operated in a manner similar to that described in the foregoing embodiment. Thus, details thereof are not repeated hereinafter.

Differences between the syringe and needle assembly 200 of the present embodiment and the syringe and needle assembly 100 of the foregoing embodiment include that an elastic sealing ring 240 sleeved on the pushing portion 232 of the plunger rod 230a entirely surrounds an outer surface of each of the elastic hooks 234, such that the elastic hooks 234 can be securely and reliably engaged with the engaging structure 222 in an engaged position by the elastic sealing ring 240. Furthermore, the elastic sealing ring 240 has an extending portion 260 located between the elastic hooks 234. In the present embodiment, the extending portion 260 is, for example, an elastic filling pillar, and extends out from an inner wall (or inner bottom wall) of the elastic sealing ring 240 and forms an integral structure with the elastic sealing ring 240.

In addition, the syringe and needle assembly 200 further includes an elastic filling ring 250. As shown in FIG. 4A, the elastic filling ring 250 is sleeved on the protruding portion 2241 of the hub 224 and fits snugly against the interior of the hollow barrel 210 and serves to fill in a portion of a front chamber inside the hollow barrel 210. The extending portion 260 and the elastic filling ring 250 are disposed in a manner configured to fill in spaces in the hollow barrel 210, so as to prevent an injection liquid from remaining in the spaces after injection and to reduce waste of the injection liquid.

As shown in FIG. 4A to FIG. 4B, when the plunger assembly 230 moves towards the connecting portion 212 to push the liquid in the hollow barrel 210 to be expelled through the hollow needle 226, the elastic hooks 234 of the plunger rod 230a and the elastic sealing ring 240 are stopped by an end surface (facing the plunger assembly 230) of the elastic filling ring 250. At the time, an end of the engaging structure 222 facing the plunger rod 230a is also stopped by the extending portion 260, such that the plunger assembly 230 is prevented from being unexpectedly pushed by the user to the engaging state (as shown in FIG. 4D). As shown in FIG. 4C, when the user intends to have the elastic hooks 234 engaged with the engaging structure 222, a greater force needs to be applied to push the plunger assembly 230, such that the plunger assembly 230 keeps moving towards the connection portion 212 against an elastic restoring force of the elastic filling ring 250. At the time, the plunger rod 230a and the elastic sealing ring 240 can compress the elastic filling ring 250, as shown in FIG. 4D, and thus, each of the elastic hooks 234 is tightly engaged with the engaging structure 222.

The hollow barrel 110 (or the hollow barrel 210), the needle unit 120 (or the needle unit 220), and the plunger assembly 130 (or the plunger assembly 230) may be made of plastic, high molecular polymer, metal, or other suitable materials and may be manufactured by injection molding, casting forming, or other suitable methods. The invention is not limited thereto.

In view of the foregoing, in the syringe and needle assembly 100/200 provided by the present embodiments, the plunger assembly 130/230 can be continuously moved towards the connecting portion 112/212 of the hollow barrel 110/210 after injection, such that the elastic hooks 134/234 of the plunger rod 130a/230a can engage with the engaging structure 122/222 of the needle unit 120/220, and then the needle unit 120/220 can be withdrawn into the interior of the hollow barrel 110/210, by pulling the plunger assembly 130/230 in the direction away from the connecting portion 112/212. Therefore, the used needle 126/226 of the syringe and needle assembly 100/200 is not exposed outside the hollow barrel 110/210, so as to prevent inadvertent contact with the contaminated needle, or the syringe and needle assembly 100/200 is prevented from being repeatedly used owing to human errors which may result in problems such as disease transmission. In addition, the syringe and needle assembly 100/200 of the present embodiment is engaged with the engaging structure 122/222 of the needle unit 120/220 through the plural separating elastic hooks 134/234, rather than being engaged with the engaging structure of the needle unit through the engaging hole at the middle of the annular structure as did in conventional retractable needle syringe. Therefore, each of the elastic hooks 134/234 can be smoothly and snugly engaged with the engaging structure 122/222 of the needle unit 120/220 through a favorable elastic deformation capability thereof Moreover, a rigidity of the material of the elastic hooks 134/234 is preferably greater than a rigidity of the material of the engaging structure 122/222, such that more secure engaging effect is achieved. In addition, the elastic hooks 134/234 separating from each other are easy to be demolded during manufacturing, and thus problems such as excessive manufacturing tolerance can be prevented. Therefore, engaging state provided by the embodiments of the invention is tight, secure, and highly reliable compared to conventional technology.

It will be apparent to those skilled in the art that various modifications and variations can be made to the structure of the present invention without departing from the scope or spirit of the invention. In view of the foregoing, it is intended that the present invention cover modifications and variations of this invention provided they fall within the scope of the following claims and their equivalents.

What is claimed is:

1. A syringe and needle assembly, comprising:
an elongated hollow barrel, comprising proximal and distal ends, an interior wall, and openings at the proximal and distal ends, wherein the distal end defines a connecting portion at the opening of the distal end;
a needle unit, comprising a hollow needle, a hub and an engaging structure connected to the hub, wherein the hub having an interior passageway is removably held in the opening of the distal end of the elongated hollow barrel through the connecting portion, the engaging structure is located in the elongated hollow barrel, and the hollow needle is mounted in the hub and the engaging structure; and
a plunger assembly being axially displaceable within the elongated hollow barrel, and comprising an elongated plunger rod, a pushing portion formed in the front end of the plunger rod and an operating portion formed in the rear end of the plunger rod, wherein the pushing portion facing the connecting portion has a plurality of elastic hooks, and the elastic hooks are separated from each other and are disposed correspondingly to the engaging structure.

2. The syringe and needle assembly as claimed in claim 1, wherein the engaging structure includes an interior passageway corresponding to the interior passageway of the hub, and a first engaging surface which faces the connecting portion, and the elastic hooks have opposed hook-shaped ends which are spared from each other by a distance less than a diameter of the first engaging surface.

3. The syringe and needle assembly as claimed in claim 2, wherein a protruding portion extends radially outwardly from a bottom surface of the hub to connect to the engaging structure.

4. The syringe and needle assembly as claimed in claim 2, wherein the first engaging surface is perpendicular to a moving direction of the plunger assembly.

5. The syringe and needle assembly as claimed in claim 2, wherein the first engaging surface is an annular flat surface.

6. The syringe and needle assembly as claimed in claim 2, wherein the engaging structure further comprises a first guiding inclined surface, the first guiding inclined surface extends downwards along an edge of the first engaging surface, and a radial width of the engaging structure gradually reduces in a direction away from the connecting portion.

7. The syringe and needle assembly as claimed in claim 6, wherein the engaging structure is a truncated cone structure.

8. The syringe and needle assembly as claimed in claim 6, wherein each of the elastic hooks comprises an elastic arm portion and an engaging portion, a spacing distance between the engaging portions is less than a radical width of the engaging structure, and when each of the engaging portions is in contact with the engaging structure and the plunger assembly keeps moving towards the connecting portion, each of the elastic arm portions is elastically deformed outwardly until each of the engaging portions crosses over the engaging structure and fully engages with the first engaging surface of the engaging structure.

9. The syringe and needle assembly as claimed in claim 8, wherein each of the engaging portions has a second guiding inclined surface and a second engaging surface, and when each of the second guiding inclined surfaces is in contact with the engaging structure and the plunger assembly keeps moving towards the connecting portion, each of the elastic hooks is deformed smoothly until cross over and to engage with the engaging structure as guided by the second guiding inclined surface together with the first guiding inclined surface of the engaging structure.

10. The syringe and needle assembly as claimed in claim 9, wherein a number of the elastic hooks is two, and the two elastic hooks are respectively located at two opposite sides of the engaging structure.

11. The syringe and needle assembly as claimed in claim 9, wherein size of the second engaging surface of the elastic hook is greater than or equal to size of the first engaging surface of the engaging structure.

12. The syringe and needle assembly as claimed in claim 2, wherein a rigidity of the material of the elastic hooks is greater than a rigidity of the material of the engaging structure.

13. The syringe and needle assembly as claimed in claim 1, wherein the plunger assembly is adapted to move from a separating state towards the connecting portion to a first position for pushing a liquid contained in the elongated hollow barrel to pass through the hollow needle, the plunger assembly is adapted to move towards the connecting portion to a second position such that the elastic hooks of the plunger rod are fully engaged with the engaging structure, the second position is located between the first position and the connecting portion, and the plunger assembly is adapted to move in a direction away from the connecting portion and an associated force is thus applied to the needle unit after the elastic hooks are engaged with the engaging structure, such that the needle unit is drawn back into the elongated hollow barrel.

14. The syringe and needle assembly as claimed in claim 2, comprising an elastic sealing ring, wherein the elastic sealing ring is sleeved on the pushing portion of the plunger rod and fits snugly against an interior of the elongated hollow barrel.

15. The syringe and needle assembly as claimed in claim 14, wherein each of the elastic hooks is at least partially located outside the elastic sealing ring.

16. The syringe and needle assembly as claimed in claim 14, wherein an outer surface of each of the elastic hooks is entirely surrounded by the elastic sealing ring.

17. The syringe and needle assembly as claimed in claim 15, wherein the elastic sealing ring has an extending portion located between the elastic hooks to surround the outer peripheral surface of the elastic hooks, and the elastic hooks pass through the extending portion to at least partially locate outside the extending portion.

18. The syringe and needle assembly as claimed in claim 3, comprising an elastic filling ring, the elastic filling ring being sleeved on the protruding portion of the hub and fitting snugly against the interior of the elongated hollow barrel.

19. The syringe and needle assembly as claimed in claim 16, wherein the elastic sealing ring further comprises an extending portion located between the elastic hooks, the extending portion extends out from an inner wall of the elastic sealing ring and forms an integral structure with the elastic sealing ring.

20. The syringe and needle assembly as claimed in claim 1, wherein the plunger assembly further comprises a stopping portion connected to the plunger rod.
